(19) United States
(12) Patent Application Publication
(10) Pub. No.: US 2005/0032058 A1
(43) Pub. Date: Feb. 10, 2005

(54) Facile detection of polycythemia vera

(76) Inventors: Kenichi Takeshita, New York, NY (US); Manuel Macapinlac, Forest Hills, NY (US); Delin Chen, New York, NY (US)

Correspondence Address: KLAUBER & JACKSON, 411 HACKENSACK AVENUE, HACKENSACK, NJ, 07601, US

(21) Appl. No.: 10/485,037

(22) PCT Filed: Jul. 26, 2002
(86) PCT No.: PCT/US02/23938

Related U.S. Application Data
(60) Provisional application No. 60/307,946, filed on Jul. 26, 2001.

(51) Int. Cl. C12Q 1/68; C12P 19/34

(52) U.S. Cl. 435/6; 435/91.2

(57) ABSTRACT

A facile means of diagnosing polycythemia vera is provided by detecting expression of the DLX7 gene in a sample of a hematopoietic cell from an individual. Increased expression of DLX7 may also be used to diagnose acute myeloid leukemia. A method is provided for treating patients suffering from polycythemia vera or acute myeloid leukemia using agents that inhibit or prevent expression of the DLX7 gene or that inhibit or prevent any downstream effects of DLX7 gene expression. Examples of the agents useful in this treatment are DLX7 antisense oligonucleotides and decoy therapy directed at binding the DLX7 gene product and preventing its contribution to the pathogenesis of polycythemia vera.

### FIELD OF THE INVENTION

This invention relates to the field of diagnosis of hematologic disorders, in particular, methods of detection of polycythemia vera (PV) and acute myeloid leukemia (AML) through monitoring of DLX7 gene expression. Methods of treating patients suffering from polycythemia vera and acute myeloid leukemia are also covered.

### BACKGROUND OF THE INVENTION

Polycythemia vera (PV) is a hematologic disorder characterized by increased red cell mass in the setting of a normal or less-than-normal serum erythropoietin level. It is one of the myeloproliferative disorders and is considered to be a non-malignant, clonal disorder. One important cellular feature of this disease is the presence of circulating red cell progenitor cells that have been variously termed “erythropoietin-independent progenitor cells,” “endogenous progenitor cells,” or “hypersensitive progenitor cells” (Reid (1987) Blood Reviews 1: 133-40). These terms refer to the ability of the PV erythroid progenitor cells to form erythroid colonies (CFU-E) in the absence of added erythropoietin (EPO) to the culture medium. In contrast, normal erythroid progenitor cells require the addition of erythropoietin. Additional examination using serum-free systems have demonstrated that PV erythroid progenitor cells do require erythropoietin but need only greatly reduced concentrations of EPO (Dai et al. (1991) J. Clin. Invest. 87:391-6; Dai et al. (1997) Blood 89:3574-3581). Studies using serum free culture methods have demonstrated that PV erythroid and probably myeloid progenitors require only very small amounts of growth factors and are thus hypersensitive to growth factors such as IL-3, EPO, SCF, GM-CSF and IGF-1.

Although in some PV patients, acute leukemia develops, a major cause of morbidity in patients with PV results from thrombotic complications. Approximately 40% of patients have been reported to die of thrombotic complications, of which cerebral, coronary, pulmonary and mesenteric thrombosis account for the majority of thromboses (Fruchtman et al. (1995) In: Blood: Principles and Practice of Hematology (Handin et al., eds.), J. B. Lippincott Co., Philadelphia, pp 415-438; Rossi et al. (1998) J. Intern. Med. 244:49-53). The increased risk of thrombosis is not simply be due to reduced blood flow from erythrocytosis, because PV patients are prone to thrombotic complications even with normalized blood volume (in fact, phlebotomy may increase the risk of thrombosis (Barbui et al. (1997) Semin. Thromb. Hemost 23:455-61), indicating that additional factors, for example platelet or some other cell abnormality, play a role in thrombosis (Fruchtman et al. (1995) supra). PV is a disease distinct from familial forms of erythrocytosis, usually associated with an erythropoietin receptor (EpoR) mutation or to the abnormality of a yet unidentified gene located at 11q23 (Sergeyeva et al. (1997) Blood 89:2148-2154).

Treatment of PV includes phlebotomy to remove excess blood, and chemotherapy. PV is commonly detected by routine blood counts as an increased hematocrit, often associated with an increase in the platelet count and white cell count. This disease is currently more accurately diagnosed using a series of diagnostic tests in an algorithm to exclude other causes of increased red blood cell production and includes a number of several time-consuming, laborious and expensive tests to complete the diagnosis. Since PV is a monoclonal proliferation of hematopoietic cells (predominantly erythroid cells), one might expect that the disease is caused by a mutation in gene(s). However, no mutations have been identified. No consistent cytogenetic changes are known, although a deletion at 20q has been identified in about 20% of patients. Preliminary data on the efforts to define this deletion indicates that the extent of the deletion is different among individual patients (Gribble et al. (2000) Blood 96 (Suppl)1:3206), suggesting that the junctions of the 20q deletions are probably not important. A deleted region common to all patients studied was identified to be approximately a few hundred Mb, raising the possibility that genes involved in the pathogenesis of PV with the 20q deletion may be located in this common region. Another group has recently identified a human homologue of the Polycomb gene located at the junction of one such 20q deletion (Alvarez et al. (2000) Blood 96(Suppl.)1:4444).

Several groups have studied molecular markers (markers expressed only in PV) in part to attempt to identify the mutation(s) that cause the disease. These markers include the BCL-XL (a moderate increase in the BCL-XL compared to control) (Silva et al. (1998) N. Engl. J. Med. 338:564-71), PRV-1 (a UPAR like gene expressed in PV and in G-CSF mobilized peripheral blood stem cell preparations but not in normal bone marrow or peripheral blood) (Temerinac et al. (2000) Blood 95:2569-76), TPO (a glycosylation defect found in PV and some ET) (Moliterno et al. (1998) N. Engl. J. Med. 338:572-80), EPO-R splice variants (Chiba et al. (1997) Blood 90:97-104), SHP-1 loss (absence of SHP-1 protein in PV despite presence of normal mRNA) (Asimakopoulos et al. (1997) Oncogene 14:1215-1222; Wickrema et al. (1999) Exp. Hematol. 27:1124-32). These genes are at best called “markers,” because it is unclear whether any of these genes are involved in the primary pathogenesis of the disease.

Recently two molecular markers have been described. Spivak et al. described a glycosylation defect in the thrombopoietin receptor (U.S. Pat. Nos. 6,150,120 and 6,027,902) of patients with polycythemia vera and reported that this defect was specific to polycythemia vera. Klippel et al. reported that a gene called PRV is a specific marker of polycythemia vera (Klippel et al. (2000) Blood 95:2569-2576).

### SUMMARY OF THE INVENTION

In accordance with the present invention, a method for the diagnosis of polycythemia vera in an individual is provided by detecting the expression of the DLX7 gene in a cell from the individual. Preferably, the cell is a cell of the hematopoietic lineage, and most preferably, a peripheral white blood cell or platelet isolated from whole blood. Bone marrow cells may be used. The expression of the aforementioned gene may be carried by any means for detecting expression of a gene, such as but not limited to RT-PCR. The method may also be used to detect acute myeloid leukemia. DLX7 expression levels diagnostic for PV are elevated above the low levels of expression seen in hematopoietic cells, such as but not limited to levels detectable by Northern blotting or using about 30 or fewer PCR cycles.

A further aspect of the invention comprises a method for treating patients suffering from polycythemia vera using agents that inhibit or prevent expression of the DLX7 gene or by using agents that inhibit or prevent any downstream effects of DLX7 gene expression. Such agents and methods of inhibition or prevention of DLX7 gene expression include administration of a DLX7 antisense oligonucleotide, or oligonucleotide decoy therapy directed at binding the DLX7 gene product and preventing its contribution to the pathogenesis of polycythemia vera. Such agents and methods of inhibition or prevention of DLX7 gene expression may also be used for treating patients suffering from acute myeloid leukemia.

These and other aspects of the present invention will be better appreciated by reference to the ensuing Detailed Description taken in conjunction with the following illustrative drawings.

### BRIEF DESCRIPTION OF THE DRAWINGS

FIG. 1A shows the locations of the antisense oligonucleotides used for testing. The thickness of the line corresponds to efficacy of gene inhibition (DLX7 mRNA decrease). FIG. 1B illustrates the data showing efficacy of the antisense oligonucleotides in degrading DLX7 mRNA compared to control oligonucleotides. (AS: the thickest line from panel A; S: sense; M:Mutant; mutant=same as AS except 3 bases are changed)

FIG. 2 shows the effect of antisense DLX7 oligonucleotides (AS) and control oligonucleotides (sense, S; mutant, M) on other genes in K562 cells. Four hours after oligonucleotide treatment, the cells were harvested and assayed.

FIG. 3 depicts the results of a TUNEL apoptosis assay on antisense-treated cells (Panel B or control (no oligonucletide, panel A; sense oligonucleotide, panel C) at 24 hours after treatment. No apoptosis was seen at 4 hours after treatment (not shown).

FIG. 4 shows the effect of antisense DLX7 treatment on c-myc and GATA-1 in untreated and hemin-treated HEL and in a lung cancer cell line (A549), indicating that that the antisense oligonucleotide has biological effects (inhibition of GATA-1 and c-myc) only in erythroid cells expressing DLX7 (hemin treated HEL) but not in DLX7 negative cell lines (untreated HEL) or in DLX7-expressing non-(A549) hematopoietic cell lines. (Note: hemin treatment induces an erythroid phenotype in HEL cells).

FIG. 5 shows the expression by RT-PCR of DLX7 (upper panels) and HPRT (lower panels) in peripheral blood or bone marrow mononuclear cells of PV patients and non-PV patients. Blank (lane 1, 10); K562 positive control (lanes 2, 11); normal bone marrow (lanes 3,4); Normal peripheral blood (lanes 5, 12); mobilized stem cell preparation (for auto transplant) (lanes 13, 14); peripheral blood of PV patients (lanes 6-9, 15-18).

FIG. 6 shows the expression of DLX7 in platelets of normal and PV patients. Lane 1: PBMNC from PV pt 1; lane 2: platelets from PV pt 1; lane 3: platelets from PV pt 2; lane 4: PBMNC from normal; lane 5: platelets from normal. CD19 is a lymphocyte marker and its absence in platelet lanes indicates absence of MNC contamination.

### DETAILED DESCRIPTION OF THE INVENTION

The present inventors have found that a gene heretofore unassociated with polycythemia vera, called DLX7, is expressed in cells in patients with polycythemia vera, and is also expressed in some patients with acute myeloid leukemia. This is a gene of the homeobox transcription factor family. As will be seen in the illustrative examples below, a facile test has been developed for the detection of the expression of DLX7 mRNA in blood cells from an individual, and expression is positively correlated with the diagnosis of PV in the individual. Simply, RNA is extracted from blood cells, and, in one non-limiting method, the reverse transcriptase-polymerase chain reaction (RT-PCR) is used to detect the expression of the DLX7 gene. This is a better marker for PV than any currently available. Preferably, the test is carried out on an individual with an elevated hematocrit level (erythrocytosis), in which a diagnosis of polycythemia may be suspected, but it may be carried out on any sample. Detection of DLX7 expression may be part of a routine screening procedure involving detection of expression of the DLX7 and other genes, as may be done with a microarray (gene chip) method, but it is not so limiting.

The sequence of the DLX7 gene is set forth in SEQ ID NO: 1. Any means to identify expression of this gene, including identification of levels of DLX7 mRNA, using, for example, any PCR primers for the gene, are embraced herein. By way of non-limiting example, the primers used in the examples below are set forth as SEQ ID NO:2 and SEQ ID NO:3. These are merely non-limiting examples of primers, and others that will amplify the DLX7 gene may be used to achieve the same objective of the invention.

Detection of expression of the DLX7 gene may be performed in any manner in which mRNA levels for this gene may be detected, or its protein expression product (SEQ ID NO:4) may be detected, and the invention is not limited to any particular methodology. Preferably, RT-PCR may be used. Microarray methods, also known as gene chip technology, may also be used. Other methods that may be used to detect expression of DLX7 may include any conventional method used in molecular biology to study gene expression and may include, but is not limited to, northern blot hybridization and ribonuclease protection assays using DNA or RNA probes corresponding to the DLX7 gene sequence, protein binding assay or gel retardation assays using probes corresponding to the DNA binding sequence of the DLX7 protein, and western blotting, immunohistochemical or immunofluorescent assay using antibody directed against the DLX7 protein (SEQ ID NO:4).

The level of expression of the DLX7 gene in polycythemia vera is readily detectable and higher than the low level of DLX7 expression reported in normal hematopoietic cells (such as described by Shimamoto et al., 1997, Proc. Nat. Acad. Sci. U.S.A. 94:3245-9). For example, the level of detection of DLX7 in normal hematopoietic cells cannot be discerned using a Northern blot, yet the level expressed in PV cells is readily detectable. Furthermore, if a PCR-type assay is performed, such as under standard conditions using about 30 cycles, or fewer, expression of DLX7 in normal cells will not be detected, yet that from PV cells will be. Only at a much higher number of cycles, such as 40 or 45, does the low level of expression of DLX7 in non-PV hematopoietic cells as previously reported become detectable and apparent. Thus, as described herein, the level of expression of DLX7 as being diagnostic of PV is visibility on a Northern blot or at or below about 30 PCR cycles. The foregoing conditions are merely illustrative and non-limiting, as a skilled artisan can readily determine the values above which a diagnosis of PV may be made, and the levels below which a low level and non-diagnostic level for PV may be made, based on the teachings herein and the inventors' finding that DLX7 is expressed in PV hematopoetic cells well above the levels in normal, non-PV hematopoietic cells.

In a further embodiment, internal positive and negative controls for DLX7 may be established and used for comparative purposes to determine whether DLX7 is expressed in the level to be diagnostic for PV. In an example of a positive control, expression of a constitutively-produced protein such as actin may be concurrently determined, and the value of a ratio of actin to DLX7 expression used to establish a diagnostic PV-positive range, a negative range, and a cut-off (rule in or rule out) value. In another embodiment, a gene which is not expressed or is expressed at only low values independently of PV may be used as a negative control, to which the DLX7 expression may be compared, to establish the same aforementoined ranges. Again, these variations in the methods by which the level of DLX7 expression is measured and then correlated with a diagnosis of PV or not is well within the realm of the skilled artisan based on the teaching herein, and all such variations are fully embraced herein.

The cellular sample from an individual from which a diagnosis of PV may be made by identifying expression of the DLX7 gene include any cell type that is affected by PV. This includes but is not limited to peripheral blood cells, bone marrow cells and platelets. Other hematopoietic cells may be evaluated. Preferably, a readily-available sample that is obtainable without discomfort to the patient, such as whole blood, is a preferred source, but any cells of a hematopoietic lineage may be used, such as obtained by biopsy or aspiration, including bone marrow cells. Although the detection of DLX7 expression may be carried out on any sample, in a preferred embodiment, the sample is from an individual with an elevated hematocrit (erythrocytosis) in which a diagnosis as to the cause of the elevation is sought.

The DLX7 expression diagnostic test for PV described herein is highly accurate and precise. The test of the invention provides no false negatives or positives, and its diagnostic accuracy is high.

Based on the inventors' finding of elevated expression of the DLX7 gene in PV and acute myeloid leukemia (AML), a method for PV therapy is provided based upon inhibiting the elevated expression of DLX7 in hematopoietic cells of a patient, or preventing any downstream effects of the DLX7 gene product. Thus, therapies directed against DLX7 mRNA and toward increasing DLX7 mRNA turnover are provided by DLX7 antisense oligonucleotide therapy, which may be exposed to hematopoietic cells of the patient by conventional methods of administration of antisense compounds. Thus the invention is directed to DLX7 antisense oligonucleotide compositions, pharmaceutical compositions containing them, and methods for treating PV and AML by their administration, and toward the corresppondin use of e.g. DLX7 antisense oligonucleotide compositions for the preparation of a pharmaceutical composition or medicament for use in the treatment of PV and acute myeloid leukemia.

The inhibiting of downstream effects of the DLX7 gene product may be therapeutically addressed by use of decoy oligonucleotides (see, for example, D'Acquisto et al., 2000, Gene Ther 7:1731-7). Such decoy oligonucleotides may be prepared for binding the DLX7 gene product, and preventing any unwanted activity towards its natural targets. While Applicants are not required to disclose the theory by which the invention operates and are in no way bound thereby, such decoy oligonucleotides are believed to inhibit transcriptional activation by the DLX7 gene product by preventing DLX7 from binding to DNA targets which in turn increase expression of genes which contribute to the PV phenotype. This aspect of the invention includes DLX7 gene product-binding decoy oligonucleotide compositions, pharmaceutical compositions containing them and methods for their administration for the treatment of PV and acute myeloid leukemia, and the concomitant use of such decoy oligonucleotide compositions for the preparation of pharmaceutical compositions or medicaments for the treatment of PV or AML.

The following examples are presented in order to more fully illustrate the preferred embodiments of the invention. They should in no way be construed, however, as limiting the broad scope of the invention.

### EXAMPLE 1

### DLX7 gene is a Transcription Factor Gene Expressed in Erythroid Cells

A homeobox gene called DLX7 (Genbank accession U73328) [Nakamura S, Stock D W, Wydner K L, Bollekens J A, Takeshita K, Nagai B M, Chiba S, Kitamura T, Freeland T M, Zhao Z, Minowada J, Lawrence J B, Weiss K M, Ruddle F H. Genomic analysis of a new mammalian distal-less gene: DLX7. Genomics 38 314-324, 1996] was cloned and characterized. This gene was initially isolated as a part of a study to identify homeobox gene families expressed in lymphoid and non-lymphoid leukemia cell lines. In this screening, genes from the Drosophila distal-less family (called DLX in humans) constituted the largest proportion of the divergent homeobox genes (i.e., not of the classical HOX family) expressed in hematopoietic cells. Subsequent work by us and others identified six members of the human DLX family, located as pairs of genes at three locations in the genome.

The DLX7 gene was selected for further study, because it is a member of the DLX gene family, many members of which are expressed in hematopoietic cells, and because it was expressed at highest levels in human erythroid cell lines such as K562, TF1 and hemin-treated HEL cells. The DLX7 gene was also expressed in about 15% of bone marrow cells from patients with AML. On the other hand, most non-erythroid myeloid cell lines express no DLX7 or low levels of DLX7. The complete cDNA has been cloned and its human chromosomal location mapped, placing it about 10 kB downstream of DLX3 gene.

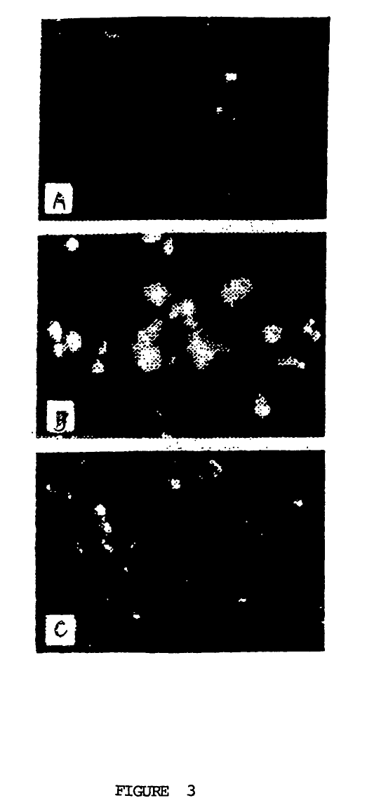

The studies herein show that the DLX7 gene confers growth factor “independence” in growth factor dependent cell lines. To further investigate the function of DLX7 gene, the DLX7 cDNA was over-expressed in the BaF3 cell line, a murine L3-dependent cell line. DLX7 cDNA transfection resulted in reduced growth factor requirements in three separate clones (FIG. 3), while the negative controls remained growth factor dependent. However, it is notable that the DLX7 transfectants grew more slowly in the absence of IL-3 compared to growth in the presence of IL3.

A more detailed examination of the growth factor requirements in which varying amounts of IL3 were added indicated that the DLX7 transfectants have reduced requirements for IL3. A comparison with the BCR-ABL transfectant demonstrates that the DLX7 gene is not as effective as the BCR-ABL oncogene in permitting growth in the absence of added IL3. This suggests that DLX7 is not a true oncogenic gene, in agreement with the finding that DLX7 gene is also not tumorigenic by the NIH-3T3 assay.

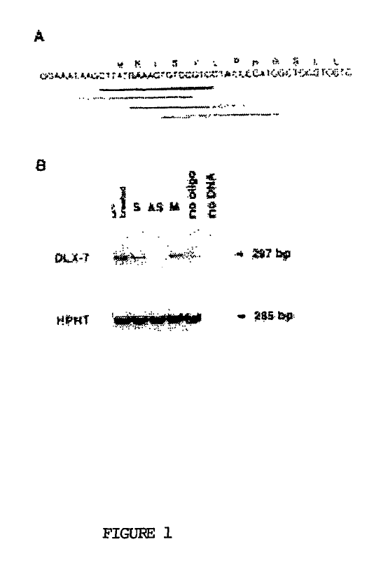

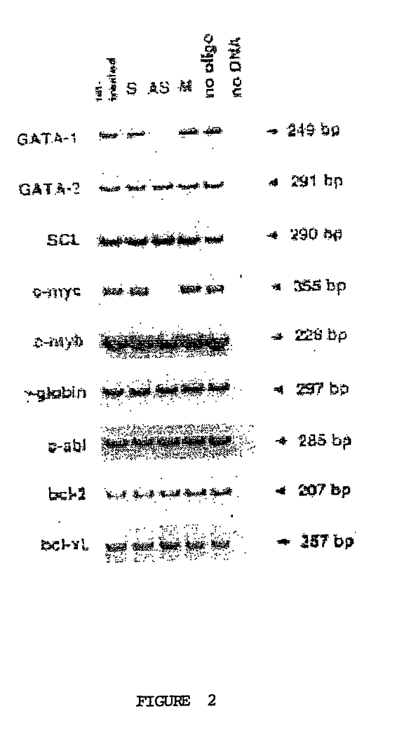

Antisense oligonucleotide directed against DLX7 gene. The possible function of the DLX7 gene was also investigated by inhibiting its expression using antisense oligonucleotides. FIG. 1 demonstrates that the antisense oligonucleotide used here reduces DLX7 mRNA in a sequence-specific manner, with subsequent degradation of the target mRNA. Further examination of antisense-treated cells showed simultaneous reduction in c-myc and GATA-1 mRNA (FIG. 2), while the mRNA for other genes (SCL, c-MYB, γ-globin, c-ABL, BCL2, BCL-XL) remained unchanged (not shown) at 3 hours after treatment.

Nuclear run-off studies on cells at 6 hours after treatment, prior to any morphological changes or detectable apoptosis, showed that antisense treatment caused decreased transcription of exons 1 and 2 of the c-myc, while unexpectedly the GATA-1 gene transcription was unchanged (not shown). This was accompanied by a decreased level of free E2F (i.e., decreased level of transcriptionally active E2F, not bound to Rb). Actinomycin D chase studies showed that c-myc mRNA stability was unchanged with antisense treatment, while the decreased GATA-1 mRNA level caused by the antisense treatment was due to increased instability.

The mRNA changes discussed above occur within a few hours of antisense treatment, prior to any morphological change. At 3-6 hours, the cells appear morphologically normal, but by 24 hours after antisense treatment, cell apoptosis is observed in antisense treated cells but in negative controls (FIG. 3).

We also studied whether the effect of the antisense oligonucleotide depended on the cell context, by treating additional erythroid and non-hematopoietic cells. The same antisense oligonucleotide in a non-hematopoietic cell line (lung cancer line A549) inhibited DLX7 expression but had no effect on the ectopically expressed GATA-1 mRNA (see FIG. 10; A549 experiment). This also indicates that the increased instability of GATA-1 mRNA seen in the prior experiment of FIG. 8 is not likely due to be due to direct effects of the antisense oligonucleotide (i.e., direct binding of the antisense oligonucleotide to the GATA-1 mRNA), because while DLX7 mRNA disappears, the ectopically expressed GATA-1 mRNA persists in this lung cancer cell line.

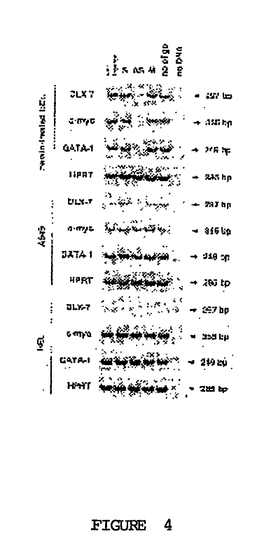

Human erythroleukemia cell line HEL normally expresses barely detectable levels of DLX7 mRNA, while hemin-treatment induces erythroid phenotype and DLX7 gene expression. DLX7 antisense treatment of untreated HEL cells had no effect on GATA-1 and c-myc genes. Antisense-oligonucleotide treatment of hemin-treated HEL cells (FIG. 4) caused associated decreases in c-myc mRNA and GATA-1 mRNA. As indicated above, the non-hematopoietic cell line expressing DLX7 (A549, lung adenocarcinoma) showed a reduction in the DLX7 mRNA but had no effect on c-myc or cell growth. These data suggest that the secondary effects of inhibition of DLX7 expression by antisense oligonucleotides depend on the cellular context.

DLX7 gene in polycythemia vera (PV) patients: We hypothesized that DLX7 may be involved in polycythemia vera, based on the findings that (1) DLX7 is expressed at high levels in erythroid cell lines but not in normal bone marrow cells or mobilized peripheral stem cell preparations, (2) antisense oligonucleotide to DLX7 mRNA caused secondary changes in gene expression (GATA-1, c-myc) and apoptosis in DLX7-expressing erythroid cell lines but not in a DLX7-expressing lung cancer cell line, (3) DLX7 gene transfection conferred reduced growth factor requirements (BaF3 cell transfection studies, see above), and (4) DLX7 gene transfection in BaF3 induced cell aggregation (see below).

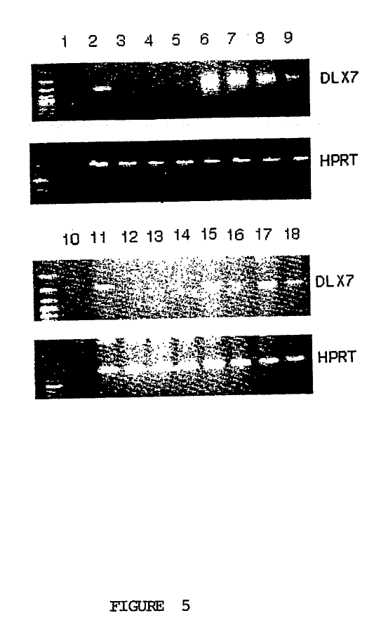

We have studied DLX7 expression in a small number of PV patients thus far, as well as bone marrow cells and peripheral blood cells of normal patients, and peripheral blood stem cells of patients undergoing mobilization for autologous transplantation (see FIG. 5). The key to the figure is: Blank (lanes 1, 10); K562 positive control (lanes 2, 11), normal bone marrow (lanes 3,4), normal peripheral blood (lanes 5, 12), mobilized stem cell preparation (for auto transplant) (lanes 13,14); and peripheral blood of PV patients (lanes 6-9, 15-18). The data is summarized in the following table.

TABLE 1
Summary of DLX7 expression in peripheral blood and
bone marrow mononuclear cells

| | Number of | |
|---|---|---|
| | subjects | Expression |
| Source of RNA | studied | of DLX7 |
| Normal bone marrow mononuclear cells | 3 | 0/3 |
| Normal peripheral blood mononuclear cells | 3 | 0/3 |
| Peripheral “stem cell” preparation | 3 | 0/3 |
| mobilized with G-CSF | | |
| Mononuclear cells from peripheral blood of | 25 | 25/25 |
| polycythemia vera patients | | |
| Mononuclear cells from peripheral blood of | 4 | 0/4 |
| patients with | | |
| hypoxia-induced erythrocytosis | | |
| Bone marrow of newly diagnosed adult AML | 15 | 2/15 |

Expression was scored as positive if a PCR band could be clearly seen on ethidium stained gel (all positive patients were equivalent to lane 9 in FIG. 5 or better in intensity). The expression was scored as negative if no band was seen or if a band was visible only after hybridization of the PCR product to a radiolabelled probe.

As a comparison, another member of the DLX family, DLX1, which is expressed in most myeloid cell lines and in more than 50% of AML patients, is not expressed in the peripheral blood mononuclear cells of any of the PV patients studied so far.

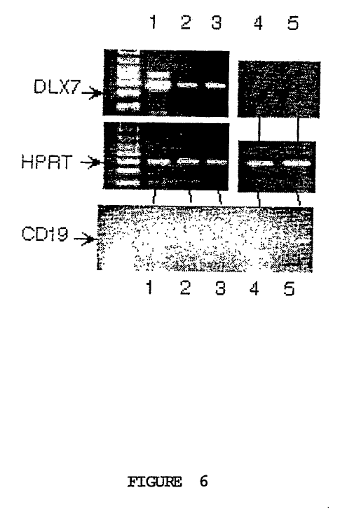

Expression of DLX7 in platelets in PV: Because PV is a multi-lineage disease and because a major cause of morbidity is thrombosis, we determined the expression of DLX7 in platelets of PV patients. Although platelets are non-nucleated, they contain small amounts of RNA, especially the newly formed reticulated platelets. We used the RT-PCR assay to determine expression in purified platelets from normals and from PV patients. DLX7 mRNA is absent in the platelets of purified from normals but is readily detectable in the platelets of 2/2 PV patients studied so far (see FIG. 6) using platelets purified by a method that eliminated mononuclear cell contamination.

FIG. 6 shows the expression of DLX7 in platelets of normal and PV patients. Lane 1 PBMNC from PV patient 1; Lane 2 platelet from PV patient 1; Lane 3 platelet from PV patient 2; lane 4 PBMNC from normal; lane 5, platelet from normal. CD19 is a lymphocyte marker and its absence in platelet lanes indicate absence of MNC contamination

While the invention has been described and illustrated herein by references to the specific embodiments, various specific material, procedures and examples, it is understood that the invention is not restricted to the particular material combinations of material, and procedures selected for that purpose. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

1. A method for the diagnosis of polycythemia vera in an individual comprising the steps of
(a) obtaining a sample of at least one hematopoietic lineage-derived cell of from the individual;
(b) detecting expression of a DLX7 gene in the cell; and
(c) correlating the expression of the DLX7 gene with a diagnosis of polycythemia vera in the individual.

2. The method of claim 1, wherein the hematopoietic lineage-derived cell is selected from the group consisting of a peripheral blood mononuclear cell, a platelet and a bone marrow cell.

3. The method of claim 1, wherein the expression of DLX7 is carried out using RT-PCR.

4. A method for the diagnosis of acute myeloid leukemia in an individual comprising the steps of:
(a) obtaining a sample of at least one hematopoietic lineage-derived cell of from the individual;
(b) detecting expression of a DLX7 gene in the cell; and
(c) correlating the expression of the DLX7 gene with a diagnosis of acute myeloid leukemia in the individual.

5. The method of claim 4, wherein the hematopoietic lineage-derived cell is selected from the group consisting of a peripheral blood mononuclear cell, a platelet and a bone marrow cell.

6. The method of claim 4, wherein the expression of DLX7 is carried out using RT-PCR.

7. A method for treating patients suffering from polycythemia vera, the method comprising treatment with agents that inhibit or prevent expression of the DLX7 gene.

8. A method for treating patients suffering from polycythemia vera, the method comprising treatment with agents that inhibit or prevent any downstream effects of DLX7 gene expression.

9. The method of claim 7, wherein the method further comprises administration of a DLX7 antisense oligonucleotide.

10. The method of claim 8, wherein the method further comprises administration of a DLX7 antisense oligonucleotide.

11. The method of claim 7, wherein the method further comprises administration of oligonucleotide decoy therapy directed at binding the DLX7 gene product and preventing its contribution to the pathogenesis of polycythemia vera.

12. The method of claim 8, wherein the method further comprises administration of oligonucleotide decoy therapy directed at binding the DLX7 gene product and preventing its contribution to the pathogenesis of polycythemia vera.

13. A method of treating a patient suffering from acute myeloid leukemia, the method comprising treating the patient with an agent that inhibit or prevent expression of the DLX7 gene.

14. A method of treating a patient suffering from acute myeloid leukemia, the method comprising treating the patient with an agent that inhibit or prevent a downstream effect of DLX7 gene expression.

15. The method of claim 13, wherein the method further comprises administration of a DLX7 antisense oligonucleotide.

16. The method of claim 14, wherein the method further comprises administration of a DLX7 antisense oligonucleotide.

17. The method of claim 13, wherein the method further comprises administration of oligonucleotide decoy therapy directed at binding the DLX7 gene product and preventing its contribution to the pathogenesis of polycythemia vera.

18. The method of claim 14, wherein the method further comprises administration of oligonucleotide decoy therapy directed at binding the DLX7 gene product and preventing its contribution to the pathogenesis of polycythemia vera.

19. (canceled)


SEQUENCE LISTING (4 sequences)

SEQ ID NO: 1 - DNA, 1403 bp, homo sapiens
ctaaagccgcaggcgccgcggtaccctggctgtggccctcggcgctttcttcctagggtcacaggacccatacgagtgggagctccctgggagcagaactgcgtcttgtatcacctggcgcggtgaacgtgggggttgaaacgctccacgcggaaggtagagggcaggggccaagggggcgatcctggtggctgcgctttttgctatttgctgccgacggcatgcagacgagatgcaaataagcttatgaaactgtccgtcctaccccatcgctccctcctcgcccccaacaccgtgttgtgctgcccaccagactcggagaagccgcggctgtctccggaaccctccgagcggcgccctcaggccgccgccaaaaagctccgcaagccgaggaccatctactccagcctgcagctgcagcacctaaaccagcgtttccagcacacgcagtacctggcgctgcccgagagggcccagctggcagcgcagctcggcctcacccagacccaggtaaagatctggtttcagaacaaacgctccaagtataagaagctcctgaagcagaattctgggggccaggaaggggacttccctgggaggaccttctctgtgtctccctgctccccacccctcccctacctctgggatctacccaaggcagggaccctgcccaccagtggctatggcaacagctttggaccctggtatcagcatcactcctcagatgtcctgtcgcctcagatgatgtgaatctggggaagggcgggtcaggcccacagccttcctgcaaagcccaggacccaggcagtccacctgcaccccttctgggctgggaggaaaccagctccagatgggttttctctggaggacaagcagttagaggagaaaaggggaatggagcagagcctgtacccctaaccctagcagctaaatcaaggacctcagccttatataatcattgtccccaccactaccatggactggacaccttcactccagctggacaaagactctggagagagagccattggctggagttgagactgtccccagaacccttggtcttgccactcccccactccttcttccctctctccctttctcctctccctgctttcttgaaaaggactgaatcgccactacagcctgggtgcaaaatcagcaagaaacattgagtatttttttttctttgtatgcctttggccttgcacaacccatttgtgagcaaaagcagaagtggaccaccatcagctcccacccacccagcgatttttccttggaggtcagcccgttacccccataactgatttacctacttaccatactgggaggtagaagagatgcagagaaatgtggaatttgtggacctatgggtaatttatgctttcctcctaaaaaaaaaa

SEQ ID NO: 2 - DNA, 18 bp, Artificial Sequence
  primer
ctccagcctgcagctgca

SEQ ID NO: 3 - DNA, 22 bp, Artificial Sequence
  primer
ccaaagctgttgccatagccac

SEQ ID NO: 4 - protein, 167 aa, homo sapiens [sequence omitted]